SEQUENCE LISTING (4 sequences)

SEQ ID NO: 1 - protein, 607 aa, synthetic construct
MGSSHHHHHHSSGLVPRGSHMDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPAQVVAIASNNGGKQALETVQRLLPVLCQDHGLTPAQVVAIASNGGGKQALETVQRLLPVLCQAHGLTPDQVVAIASHDGGKQALETVQRLLPVLCQDHGLTPAQVVAIASNGGGKQALETVQRLLPVLCQAHGLTPDQVVAIASNNGGNEQALETVQRLLPVLCQAHGLTPAQVVAIASNGGGKQALETVQRLLPVLCQDHGGGSGGPGMVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGITLGMDELYK

SEQ ID NO: 2 - DNA, 33 bp, synthetic construct
  source 1..33: mol_type = other DNA
atgttgcatatggatctacgcacgctcggctac

SEQ ID NO: 3 - DNA, 67 bp, synthetic construct
  source 1..67: mol_type = other DNA
atgttgggatccatgttgcccggggccgccagagccgcccccatgatcctgacacaaaacaggcaac

SEQ ID NO: 4 - protein, 5 aa, synthetic construct
GGSGG

(19) United States
(12) Patent Application Publication
(10) Pub. No.: US 2025/0290120 A1
(43) Pub. Date: Sep. 18, 2025

(54) DNA-PROTEIN-POLYMER-METAL CATION COMPLEX, DNA IMAGING METHOD USING THE SAME, AND SEM-BASED DNA DETECTION METHOD

(71) Applicants: SOGANG UNIVERSITY RESEARCH & BUSINESS DEVELOPMENT FOUNDATION, Seoul (KR); RESEARCH & BUSINESS FOUNDATION SUNGKYUNKWAN UNIVERSITY, Suwon-si (KR)

(72) Inventors: Kyubong JO, Seoul (KR); Jung Heon LEE, Seoul (KR); Chanyoung NOH, Seoul (KR); Priyannth RAMASAMI SUNDHARBAABU, Gyeonggi-do (KR); Junhyuck CHANG, Gyeonggi-do (KR)

(73) Assignees: SOGANG UNIVERSITY RESEARCH & BUSINESS DEVELOPMENT FOUNDATION, Seoul (KR); RESEARCH & BUSINESS FOUNDATION SUNGKYUNKWAN UNIVERSITY, Suwon-si (KR)

(21) Appl. No.: 19/223,737

(22) Filed: May 30, 2025

Related U.S. Application Data
(63) Continuation-in-part of application No. 18/583,491, filed on Feb. 21, 2024.
(63) Continuation-in-part of application No. 19/081,184, filed on Mar. 17, 2025.

(30) Foreign Application Priority Data
Dec. 1, 2023 (KR) 10-2023-0172725
Mar. 15, 2024 (KR) 10-2024-0036772

(51) Int. Cl. C12Q 1/6825; C12Q 1/34; C12Q 1/6806

(52) U.S. Cl. CPC C12Q 1/6825; C12Q 1/34; C12Q 1/6806; G01N 2333/922

(57) ABSTRACT

Disclosed are a DNA-protein-polymer-metal cation complex, a DNA imaging method using the same, and a scanning electron microscopy (SEM)-based DNA detection method. The DNA-protein-polymer-metal cation complex disclosed herein enables real-time imaging of DNA molecules of various shapes and lengths with improved resolution by using SEM even at high magnifications, and improves DNA detection, thereby further enhancing DNA optical mapping.

### CROSS-REFERENCE TO RELATED APPLICATION

This application is a continuation-in-part of U.S. patent application Ser. No. 18/583,491, filed on Feb. 21, 2024, which claims priority from Korean Patent Application No. 10-2023-0172725 filed Dec. 1, 2023, and U.S. patent application Ser. No. 19/081,184, filed on Mar. 17, 2025, which claims priority from Korean Patent Application No. 10-2024-0036772 filed Mar. 15, 2024, the entire disclosures of which are incorporated herein by reference.

### INCORPORATION BY REFERENCE OF SEQUENCE LISTING

The instant application contains a Sequence Listing which has been filed electronically in xml format and is hereby incorporated by reference in its entirety. Said xml file, created on May 28, 2025, is named Q309694_SEQLIST_ST26.xml and is 5.2 KB in size.

### BACKGROUND

### 1. Field

Disclosed are a DNA-protein-polymer-metal cation complex, a DNA imaging method using the same, and a scanning electron microscopy (SEM)-based DNA detection method.

### 2. Description of the Related Art

DNA imaging plays a central role in genomics. Various microscopy-based techniques have been employed for this purpose, including transmission electron microscopy (TEM), fluorescence microscopy (FM), and scanning electron microscopy (SEM).

TEM offers the advantage of enabling detailed observation of DNA fine structures; however, to overcome DNA's low electron scattering properties, imaging typically employs heavy metal salts such as uranyl acetate combined with shadow casting techniques. Consequently, observing DNA molecules via TEM involves time-consuming and complex procedural steps.

Fluorescence microscopy (FM) is widely utilized due to its simplicity, accessibility, and live imaging capabilities; however, it has the fundamental limitation of insufficient resolution, making it difficult to accurately observe DNA molecules at the nanometer scale. Another important requirement for DNA imaging is compatibility with microfluidic devices and chemically functionalized surfaces. While images obtained through FM often show well-aligned and parallel stretching of DNA, TEM struggles to achieve this due to alignment issues with carbon film-coated metal grids, which typically result in randomly arranged DNA molecules.

Scanning electron microscopy (SEM) is being investigated as a method that can overcome the limitations of both TEM and fluorescence microscopy, as SEM can simultaneously satisfy nanometer-scale resolution and millimeter-scale observation range while offering greater flexibility for accommodating functionalized surfaces and microfluidic devices used for DNA alignment.

However, SEM faces the challenge of falling short of the detection limit required for imaging DNA molecules with a thickness of 2 nm. To address these issues, researchers have employed methods such as nanoparticle attachment and nanowire growth with scanning electron microscopy (SEM). However, these approaches have primarily been used to visualize artificially constructed DNA nanostructures and present difficulties when applied to DNA molecules themselves.

Therefore, there remains a need for DNA-protein-polymer-metal cation complexes that enable real-time imaging with enhanced resolution under high magnification for DNA molecules of various shapes and lengths via SEM, as well as improved DNA optical mapping. Consequently, there is ongoing demand for DNA imaging methods, SEM-based DNA detection methods, and DNA analysis kits utilizing these complexes.

### SUMMARY

Provided is a DNA-protein-polymer-metal cation complex that enables high-resolution, real-time imaging of DNA molecules with diverse shapes and lengths under high magnification using scanning electron microscopy (SEM), and that supports improved DNA optical mapping.

Provided is a DNA imaging method using the DNA-protein-polymer-metal cation complex.

Provided is an SEM-based DNA detection method using the DNA-protein-polymer-metal cation complex.

Additional aspects will be set forth in part in the description which follows and, in part, will be apparent from the description, or may be learned by practice of the presented embodiments of the disclosure.

According to an embodiment, the DNA-protein-polymer-metal cation complex includes: a DNA molecule; a DNA-binding protein comprising a peptide having an amino acid sequence capable of binding to the DNA molecule and having at least one functional group at at least one of the N-terminal and C-terminal of the peptide; a polymer, an anhydride thereof, or a salt thereof, which is capable of binding to the DNA-binding protein via intermolecular electrostatic interaction; and a metal cation-containing component, excluding uranium, that is capable of binding to the polymer, the anhydride thereof, or the salt thereof through interaction.

According to some embodiments, the polymer may include at least one atom selected from oxygen and nitrogen, and the at least one atom may be capable of forming a hydrogen bond with a hydrogen atom of an O—H or N—H group present in the DNA-binding protein.

According to some embodiments, the polymer, an anhydride thereof, or a salt thereof may include at least one structural unit represented by any one of Formulae 1 to 12, and have a weight average molecular weight ranging from 10 kilodaltons to 100 kilodaltons.

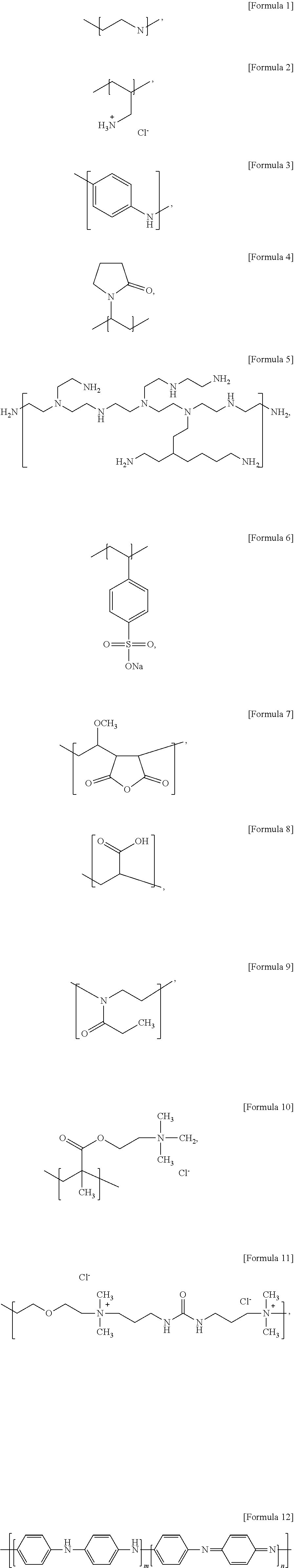

wherein in Formula 12,
m+n may be 1.

According to some embodiments, the DNA molecule may have a linear, circular, helical, supercoiled, Christmas-tree, double-stranded, or single-stranded form.

According to some embodiments, the DNA-binding protein may further include a fluorescent protein.

According to some embodiments, the DNA molecule and the DNA-binding protein may be capable of binding through one or more of electrostatic interaction, intercalation, and groove binding.

According to some embodiments, the metal cation may have a valence of +2, +3, +4, +5, or +6, and the metal cation may include at least one metal selected from lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), hafnium (Hf), tantalum (Ta), tungsten (W), rhenium (Re), osmium (Os), iridium (Ir), platinum (Pt), and gold (Au).

According to some embodiments, the metal cation may include La³⁺, Eu²⁺, Eu³⁺, Gd³⁺, Ga⁴⁺, Dy³⁺, or a combination thereof.

According to some embodiments, the complex may be a complex for staining DNA having a two-dimensional or three-dimensional structure for visualization by scanning electron microscopy (SEM).

According to some embodiments, the complex may be a complex to enable visualization of DNA in a continuous form by SEM.

According to another embodiment, the composition includes the above-described DNA-protein-polymer complex and at least one selected from a solvent, an acid, a base, and a buffer solution.

According to another embodiment, provided is a method of DNA imaging using a DNA-protein-polymer-metal cation complex, the method including: providing a silicon substrate surface-modified to have a positive charge; immobilizing and stretching DNA on a surface of the silicon substrate using a PDMS microchannel device into which the above-described composition is injected; and removing the PDMS microchannel device from the silicon substrate and imaging the immobilized and stretched DNA by SEM.

According to another embodiment, the SEM-based method for detecting DNA using a DNA-protein-polymer-metal cation complex includes: a first step of forming a nick in a target sequence motif by contacting double-stranded DNA with a nickase; a second step of generating labeled double-stranded DNA by incorporating a labeled nucleotide into the nick site of the nicked double-stranded DNA; a third step of staining the double-stranded DNA by contacting the labeled double-stranded DNA with a DNA-binding protein and a polymer, an anhydride thereof, or a salt thereof, and a metal cation-containing component excluding uranium; a fourth step of stretching and immobilizing the stained double-stranded DNA on a substrate having a functionalized surface; and a fifth step of imaging the immobilized double-stranded DNA by SEM.

According to some embodiments, the labeled nucleotide may be a nucleotide conjugated, either directly or indirectly, to a detectable label.

According to some embodiments, the detectable label may include at least one selected from metal nanoparticles, oxide particles, sulfide particles, nanoclusters, quantum dots, polymer particles, hydroxyapatite (HAp) particles, fluorescent dye particles, and magnetic particles.

According to another embodiment, provided is a SEM-based DNA analysis kit using a DNA-protein-polymer-metal cation complex including: a DNA-binding protein capable of binding to a DNA molecule through one or more of electrostatic interaction, intercalation, and groove binding; a polymer, an anhydride thereof, or a salt thereof, which is capable of binding to the DNA-binding protein via intermolecular electrostatic attraction; and a metal cation-containing component, excluding uranium, that is capable of binding to the polymer, the anhydride thereof, or the salt thereof through interaction.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic diagram of a DNA-protein-polymer-metal cation complex according to an embodiment.

FIG. 2 is schematic diagram of PDMS microchannel device according to an embodiment.

FIG. 3A shows DNA imaging result observed by FE-SEM of DNA-metal cation complex using metal cation composition (@ pH 6) prepared in Comparative Example 1.

FIG. 3B shows DNA imaging result observed by FE-SEM of DNA-metal cation complex using metal cation composition (@ pH 8) prepared in Comparative Example 2.

FIG. 4A shows DNA imaging result observed by FE-SEM of DNA-metal cation complex using metal cation composition (@ pH 6) prepared in Comparative Example 3.

FIG. 4B shows DNA imaging result observed by FE-SEM of DNA-metal cation complex using DNA-metal cation composition (@ pH 8) prepared in Comparative Example 4.

FIG. 5A shows DNA imaging result observed by FE-SEM at 50,000× magnification of DNA-polymer (or salt) complex composition prepared in Comparative Example 5.

FIG. 5B shows DNA imaging result observed by FE-SEM at 5,000× magnification of DNA-polymer (or salt) complex composition prepared in Comparative Example 6.

FIG. 6A shows DNA imaging result observed by FE-SEM at 5,000× magnification of DNA-polymer (or salt) complex composition prepared in Comparative Example 7.

FIG. 6B shows DNA imaging result observed by FE-SEM at 5,000× magnification of DNA-polymer (or salt) complex composition prepared in Comparative Example 8.

FIG. 7A shows DNA imaging result observed by FE-SEM at 5,000× magnification of DNA-polymer (or salt) complex composition prepared in Comparative Example 9.

FIG. 7B shows DNA imaging result observed by FE-SEM at 5,000× magnification of DNA-polymer (or salt) complex composition prepared in Comparative Example 10.

FIG. 8A shows DNA imaging result observed by FE-SEM at 5,000× magnification of DNA-protein-polymer (or salt)-metal cation complex composition prepared in Example 1.

FIG. 8B shows DNA imaging result observed by FE-SEM at 5,000× magnification of DNA-protein-polymer (or salt)-metal cation complex composition prepared in Example 2.

FIG. 9A shows DNA imaging result observed by FE-SEM at 50,000× magnification of DNA-protein-polymer (or salt)-metal cation complex composition prepared in Example 2.

FIG. 9B shows DNA imaging result observed by FE-SEM at 110,000× magnification of DNA-protein-polymer (or salt)-metal cation complex composition prepared in Example 2.

FIG. 10 shows DNA detection result obtained by FE-SEM at 50,000× magnification using QD-labeled circular M13mp18 double-stranded DNA solution prepared in Reference Example 1.

FIG. 11 shows DNA detection result obtained by FE-SEM at 50,000× magnification using QD-labeled circular M13mp18 double-stranded DNA solution prepared in Example 3.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of”, when preceding a list of elements, modify the entire list of elements and do not modify the individual elements of the list.

Hereinafter, the DNA-protein-polymer-metal cation complex, a DNA imaging method using the same, and a scanning electron microscopy (SEM)-based DNA detection method will be described in detail with reference to the accompanying drawings. The following descriptions are presented as examples only and do not limit the invention, which is defined solely by the scope of the appended claims.

Hereinbelow, the singular forms are intended to include the plural forms as well, unless the context clearly indicates otherwise. In the present specification, the term “comprising”, or “including”, unless otherwise specified, means that other components may be further included, not excluding other components. The terms “first”, “second”, “third”, “fourth”, and the like may be used to describe various elements but such elements should not be construed as being limited by these terms.

In the present specification, the term “combination” includes any combination, mixture, etc. among components unless specifically stated otherwise. In the present specification, “or” means “and/or” unless otherwise specified.

In the present specification, “or”, unless otherwise specified, means “and/or”.

As used herein, the term “connected” may refer to direct connection, indirect connection, or indirect communication.

As used herein, the term “metal cation-containing component, excluding uranium, that is capable of binding to a polymer, an anhydride thereof, or a salt thereof through interaction” refers to a metal cation-containing component excluding uranium that can chemically interact with the polymer (or anhydride or salt thereof) through electrostatic interaction, coordination bonding, or complex formation.

As used herein, the term “metal cation-containing component other than uranium (cation)” refers to an inorganic or organic compound that includes cations of alkali metals, alkaline earth metals, transition metals, post-transition metals, lanthanide metals, or actinide metals, excluding uranium.

As used herein, the term “nickase” refers to an endonuclease that cleaves only one strand of double-stranded DNA, i.e., introduces a nick into the double-stranded DNA.

As used herein, the term “nick” refers to a broken phosphodiester bond site in double-stranded DNA. By introducing a nick, a free 3′ strand and a free 5′ strand can be exposed at the site. That is, the nick may include a 3′ hydroxyl group and an adjacent 5′ phosphate group.

As used herein, the term “nick translation” refers to a process in which a polymerase extends the DNA strand from the 3′ hydroxyl group at the nick site while simultaneously degrading the downstream strand.

As used herein, the term “detectable label” refers to a label that can be detected through optical methods, i.e., an optical label. The detectable label may be a label detectable under scanning electron microscopy (SEM). A label may be used that is visualized as a bright contrast against the dark lines of the DNA backbone, which appear under SEM after staining.

As used herein, the term “quantum dots” refers to particulate materials used as biomolecular fluorescent labeling agents that can replace conventional fluorescent dyes, and that are used for spectroscopic observation of the behavior of proteins, DNA, metabolites, and the like within cells by conjugation to such biomolecules.

Throughout the present specification, “an embodiment”, “embodiment”, and the like mean that a particular element described in connection with the embodiment is included in at least one embodiment described in the present specification and may or may not be present in other embodiments. Furthermore, it should be understood that the described elements may be combined in any suitable manner in various embodiments.

Unless otherwise defined, all technical and scientific terms used herein have the same meanings as commonly understood by one of ordinary skill in the art to which the present application pertains. All cited patents, patent applications, and other references are incorporated herein by reference in their entirety. However, in the event of any conflict or inconsistency between the terms used in the present specification and the terms of the incorporated references, the terms used in the present specification shall prevail. While specific embodiments and implementations have been described, alternatives, modifications, variations, improvements, and substantial equivalents that are currently unanticipated or unpredictable may occur to the applicant or those skilled in the art. Therefore, the appended claims and any claims subject to amendment are intended to encompass all such alternatives, modifications, variations, improvements, and substantial equivalents.

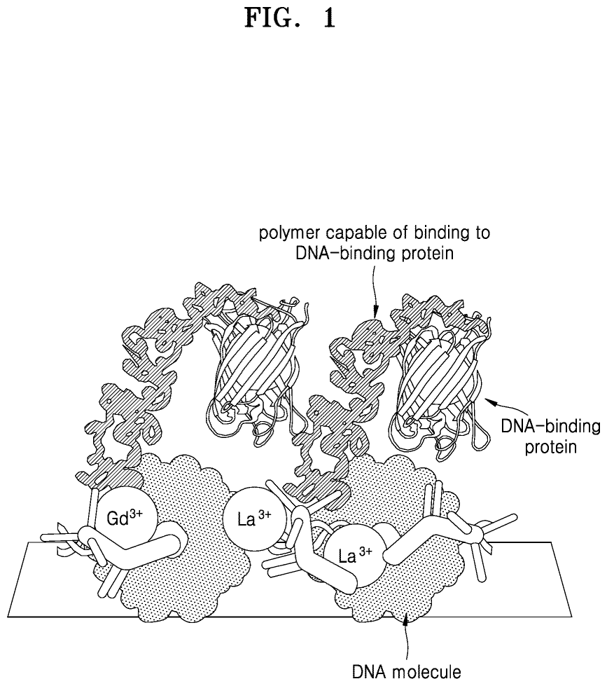

FIG. 1 is a schematic diagram of a DNA-protein-polymer-metal cation complex according to an embodiment.

Referring to FIG. 1, the DNA-protein-polymer-metal cation complex according to an embodiment includes a DNA molecule, a DNA-binding protein capable of binding to the DNA molecule, a polymer capable of binding to the DNA-binding protein, and a metal cation-containing component capable of binding to the polymer. The term “complex” refers to a structure in which two or more components are integrally combined. As used in the present specification, the DNA-protein-polymer-metal cation complex refers to a single structure in which DNA, a protein, a polymer, and a metal cation are combined.

A DNA molecule is a polymer of nucleotides, in which a phosphate group is bonded to one side of a deoxyribose backbone and one of four nucleobases is bonded to the other side. A DNA molecule constitutes the fundamental material of genes.

Any DNA molecule may be used regardless of its structure, length, or shape. The DNA molecule may have a length ranging from several kilobase pairs (kbp) to several megabase pairs (Mbp). In an embodiment, the DNA molecule may have a nanoscale thickness and a microscale length. Examples of such DNA molecules may include λ phage DNA (48.5 kb) and M13mp18 double-stranded DNA. For example, the DNA molecule may have a linear, circular, helical, supercoiled, Christmas-tree, double-stranded, or single-stranded form.

A DNA-binding protein is a protein having a DNA-binding domain and specific or non-specific affinity for a DNA molecule.

The DNA-binding protein may include a peptide having an amino acid sequence capable of binding to a DNA molecule and at least one functional group at at least one of the N-terminal and C-terminal of the peptide.

The binding between the DNA molecule and the DNA-binding protein may occur through one or more of electrostatic interaction, intercalation, and groove binding.

The amino acid sequence may include at least one selected from lysine (Lys), tryptophan (Trp), arginine (Arg), histidine (His), phenylalanine (Phe), alanine (Ala), and tyrosine (Tyr), but is not limited thereto, and any amino acid sequence usable in DNA-binding proteins may be employed.

The peptide may interact electrostatically with negatively charged DNA molecules by including positively charged amino acid residues. A peptide that binds to DNA molecules via electrostatic interaction may include an amino acid sequence containing at least one selected from arginine, histidine, and lysine.

Intercalation refers to a phenomenon in which molecules, atoms, or ions are inserted between the layers of a layered material, and a peptide containing the above amino acid sequence may be inserted into the DNA molecule. In such a case, a peptide that binds to the DNA molecule via intercalation may include an amino acid sequence containing at least one aromatic amino acid selected from phenylalanine, tyrosine, and tryptophan.

Groove binding refers to binding between the exposed bases of the DNA molecule and the peptide containing the amino acid sequence, and the peptide may bind to the DNA through groove binding.

The peptide may perform groove binding or intercalation with the DNA molecule in addition to electrostatic interaction, or may perform both intercalation and groove binding together with electrostatic interaction.

The peptide may have at least one functional group at least one of the N-terminal and C-terminal of the peptide. The functional group may include at least one selected from a thiol group, an amine group, a carboxyl group, a cysteine group, an azide group, an alkyne group, a glutaraldehyde group, and a maleimide group, but is not limited thereto, and may include any functional group usable in DNA-binding proteins. For example, the peptide may bind to the DNA molecule and thereby provide the above-described functional group to the DNA molecule.

Examples of DNA-binding proteins may include histone-like nucleoid-structural proteins such as truncated TALE (tTALE), H-NS, Cro, and HMG.

The DNA-binding protein may further include a fluorescent protein. The fluorescent protein is not particularly limited and may include any known fluorescent protein. The fluorescent protein may serve as an excellent docking site for the polymer described below. In addition, oligomerization of the fluorescent protein may enhance its binding strength with the polymer described below and may exist as an aggregate around the DNA molecule together with the polymer.

In an embodiment, the DNA-binding protein may be linked to a fluorescent protein via a linker, or the DBP and FP may be directly conjugated without a linker. Examples of DNA-binding protein-fluorescent protein conjugates may include truncated TALE ((TALE)-emGFP, H-NS-mScarlet, Cro-mNeonGreen, mNeonGreen-HMG, and truncated TALE ((TALE)-mNconGreen. Such DNA-binding protein-fluorescent protein conjugates may influence the brightness required for visualizing DNA molecules under electron microscopy and affect the stretching of DNA molecules.

In the present specification, the polymer capable of binding to the DNA-binding protein may include, in addition to the polymer itself, an anhydride thereof or a salt thereof. The salt may be an inorganic salt or an organic salt. For example, the salt may be an inorganic salt or an organic salt. For example, the salt may be a cationic inorganic salt.

In an embodiment, the polymer may include at least one atom selected from oxygen and nitrogen.

The at least one atom in the polymer may be capable of forming a hydrogen bond with a hydrogen atom of an O—H bond or an N—H bond present in the DNA-binding protein. Such hydrogen bonding corresponds to intermolecular interaction and is distinct from covalent bonding.

In an embodiment, the polymer, an anhydride thereof, or a salt thereof may be in the form of aggregates.

In an embodiment, the polymer, an anhydride thereof, or a salt thereof may include at least one structural unit represented by any one of Formulas 1 to 12:

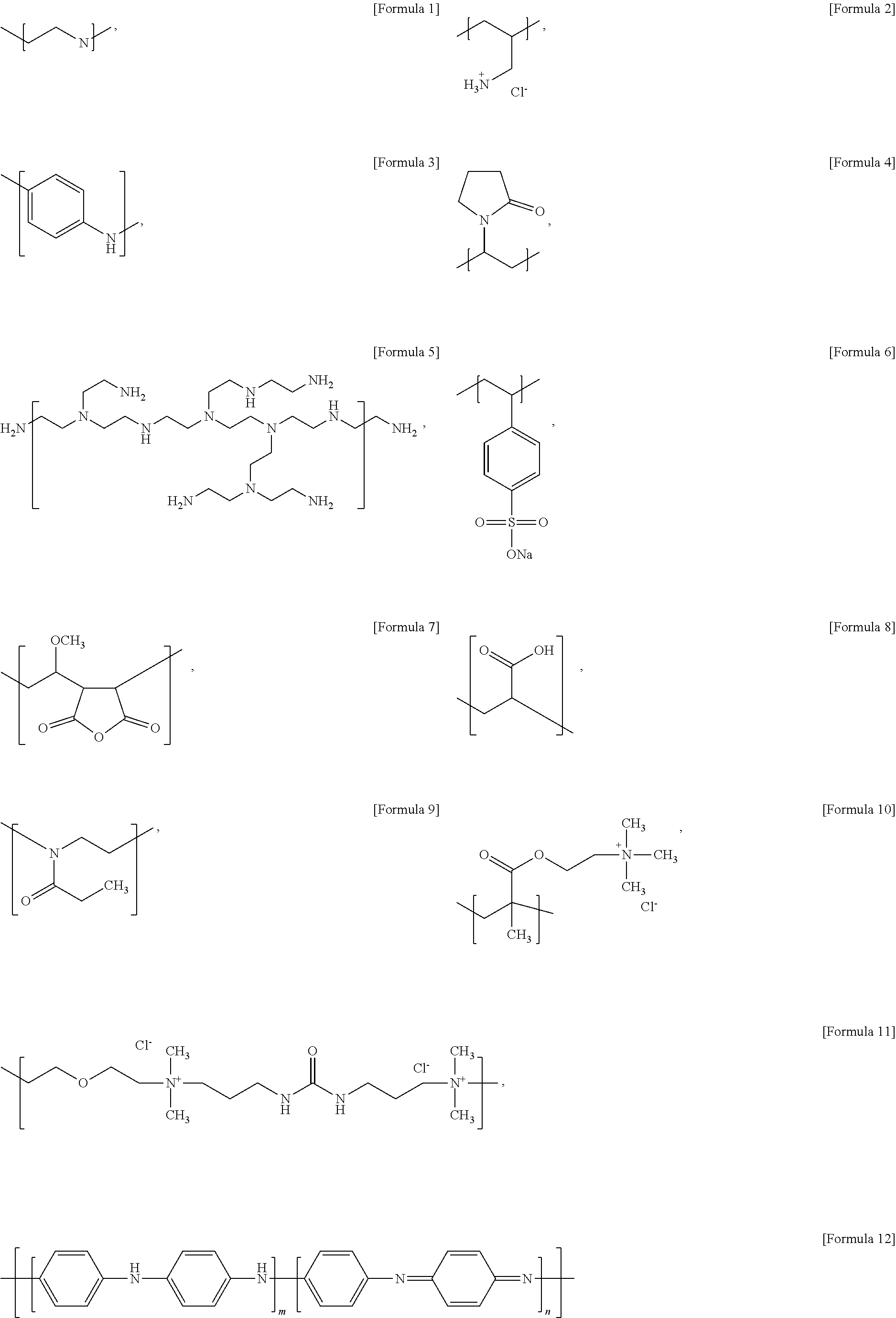

wherein in Formula 12,
m+n may be 1.

The weight average molecular weight (Mw) of the polymer, an anhydride thereof, or a salt thereof may range from about 10 kilodaltons to about 100 kilodaltons. For example, the weight average molecular weight (Mw) of the polymer, an anhydride thereof, or a salt thereof may be from about 20 kilodaltons to about 100 kilodaltons, from about 30 kilodaltons to about 100 kilodaltons, from about 40 kilodaltons to about 100 kilodaltons, or from about 40 kilodaltons to about 90 kilodaltons.

The polymer, an anhydride thereof, or a salt thereof may stain the DNA molecule black in combination with the DNA-binding protein described above, thereby contributing to the visualization of the DNA molecule through scanning electron microscopy (SEM).

The metal cation may be a metal cation excluding uranium. Specifically, the metal cation may refer to a metal cation other than uranium, which poses radiation concerns. A metal cation excluding uranium may block secondary electrons rising from a lower portion during DNA imaging by SEM, thereby enhancing imaging resolution. Unlike DNA-protein-polymer complexes that do not contain a metal cation, DNA-protein-polymer-metal cation complexes allow DNA molecules to be imaged by SEM at magnifications of 100,000× or higher with enhanced resolution, without signal loss due to electron beam damage.

In an embodiment, the metal cation may be a metal ion having a valence of +2, +3, +4, +5, or +6.

The metal of the metal cation may include at least one selected from lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), hafnium (Hf), tantalum (Ta), tungsten (W), rhenium (Re), osmium (Os), iridium (Ir), platinum (Pt), and gold (Au).

For example, the metal cation may include La³⁺, Eu²⁺, Eu³⁺, Gd³⁺, Ga⁴⁺, Dy³⁺, or a combination thereof. For example, the metal cation may include La³⁺, Gd³⁺, or a combination thereof.

The metal cation-containing component may include at least one selected from an oxide, acetate, chloride, bromide, iodide, nitrate, sulfate, carbonyl compound, or thiolate of the above-mentioned metal cations.

According to an embodiment, the complex may be used to stain DNA having a two-dimensional or three-dimensional structure for visualization by scanning electron microscopy (SEM).

According to an embodiment, the complex may be used to visualize DNA having a continuous shape by scanning electron microscopy (SEM).

According to another embodiment, a composition may include the above-described DNA-protein-polymer-metal cation complex and at least one selected from a solvent, an acid, a base, and a buffer solution. The composition may be prepared by adding the above-described DNA-protein-polymer-metal cation complex to a solvent, a buffer solution, or a mixture thereof, and further adding an acid and/or a base. The composition may further include other additives usable in the art. The contents of the solvent, acid, base, and buffer solution included in the composition may be appropriately adjusted according to the required performance. In addition, the composition may be mixed with a sample.

According to an embodiment, the DNA-protein-polymer-metal cation complex in the composition may be used to stain DNA at a pH ranging from about 6 to about 8 for visualization by scanning electron microscopy (SEM).

According to another embodiment, a DNA imaging method using a DNA-protein-polymer-metal cation complex may include: providing a silicon substrate modified to have a positive charge; immobilizing and stretching a DNA molecule on a surface of the silicon substrate using a PDMS microchannel device into which the above-described composition is injected; and removing the PDMS microchannel device from the silicon substrate and imaging the immobilized and stretched DNA using a scanning electron microscope (SEM).

A silicon substrate modified to have a positive charge serves to immobilize DNA molecules. In comparison, the surface of a glass substrate commonly used in fluorescence microscopy (FM) induces electron charging effects, and thus is not suitable for observing DNA molecules by scanning electron microscopy (SEM). In addition, a substrate having a surface coated with a conductive metal such as gold (Au) or platinum (Pt) is not suitable for observing DNA molecules under SEM, as the image of 2-nm-thick DNA molecules may appear blurred and obscured. The silicon substrate used in the present disclosure has semiconductor properties, enabling effective charge transfer.

The providing of a silicon substrate modified to have a positive charge may include: preparing a silicon substrate having a silicon oxide layer formed thereon; and bringing the silicon substrate having the silicon oxide layer into contact with an ammonium salt precursor to provide a silicon substrate modified to have a positive charge.

A silicon substrate modified to have a positive charge by an ammonium salt on a surface having a silicon oxide layer serves to immobilize the DNA molecule backbone. The thickness of the silicon oxide layer may be in a range of several nanometers to several tens of nanometers. The thickness of the silicon oxide layer may be appropriately adjusted within a range that maintains the positive charge resulting from the surface modification.

The ammonium salt precursor may include at least one selected from ammonia water, an ammonium chloride-based compound, an ammonium sulfate-based compound, an ammonium carbonate-based compound, an ammonium bicarbonate-based compound, and an ammonium acetate-based compound. For example, the ammonium salt precursor may be an ammonium chloride-based compound. For example, the ammonium salt precursor may be N-trimethoxysilylpropyl-N,N,N-trimethylammonium chloride.

A DNA imaging method according to an embodiment may further include drying the composition before removing the PDMS microchannel device from the silicon substrate.

The PDMS microchannel device is a polydimethylsiloxane microchannel device and serves to guide the DNA-protein-polymer-metal cation complex composition through the microchannel. The PDMS microchannel device stretches the DNA molecule, allowing it to appear as a well-aligned, parallel image when observed through scanning electron microscopy (SEM). After drying the DNA-protein-polymer-metal cation complex composition at room temperature for 30 minutes to 1 hour, the image is observed by scanning electron microscopy (SEM).

According to another embodiment, a scanning electron microscopy (SEM)-based DNA detection method using a DNA-protein-polymer-metal cation complex may include: a first step of forming a nick at a target sequence motif by contacting double-stranded DNA with a nickase; a second step of generating labeled double-stranded DNA by incorporating labeled nucleotides into the nick site of the nicked double-stranded DNA; a third step of staining the double-stranded DNA by contacting the labeled double-stranded DNA with a DNA-binding protein, a polymer, an anhydride thereof, or a salt thereof, and a metal cation-containing component excluding uranium; a fourth step of stretching and immobilizing the stained double-stranded DNA on a substrate having a functionalized surface; and a fifth step of imaging the immobilized double-stranded DNA using a scanning electron microscope.

The first and second steps may be for sequence-specific labeling of a DNA molecule via nick translation.

The nickase may be a nicking endonuclease.

The term “nicking endonuclease” refers to an endonuclease that recognizes a target sequence motif and forms a sequence-specific nick. The nicking endonuclease may occur naturally or may be engineered from a restriction endonuclease.

In an embodiment, the nicking endonuclease may be Nb.BbvCI, Nb.BssSI, Nb.BsmI, NbBsrDI, Nb.BtsI, Nt.AlwI, Nt.BbvCI, Nt.BsmAI, Nt.BspQI, Nt.BstNBI, or Nt.CviPII, but is not limited thereto, and any endonuclease capable of recognizing a target sequence motif and forming a nick in double-stranded DNA may be used in the method of the present disclosure. More than 200 nicking endonucleases have been studied, and some of these endonucleases are commercially available from sources such as New England Biolabs.

The target sequence motif may be a short nucleotide sequence of 8 bases or fewer. For example, Nb.BbvCI may recognize and cleave the sequence 5′-CC\hat{ }TCAGC-3′, and Nb.BssSI may recognize and cleave the sequence 5′-C\hat{ }TCGTG-3′.

Alternatively, the nickase may be an RNA-guided endonuclease.

The term “RNA-guided endonuclease” refers to an endonuclease that recognizes a target sequence motif through a guide RNA (gRNA) and forms a sequence-specific nick by forming a complex with the guide RNA. For example, an RNA-guided endonuclease may be cas9 nickase (nCAS9), and when the nickase is an RNA-guided endonuclease, the nickase may be used in combination with a guide RNA sequence partially complementary to the target double-stranded DNA.

In an embodiment, the nickase may be Nb.BbvCI, Nb.BsmI, NbBsrDI, Nb.BtsI, Nt.AlwI, Nt.BbvCI, Nt.BsmAI, Nt.BspQI, Nt.BstNBI, Nt.CviPII, nCAS9, or a combination thereof.

According to an embodiment, the labeled nucleotide may be a nucleotide conjugated, either directly or indirectly, to a detectable label. The detectable label may be conjugated to the nucleotide via a covalent bond or through a binding pair consisting of a first binding moiety and a second binding moiety.

In an embodiment, the detectable label may include at least one selected from metal nanoparticles, oxide particles, sulfide particles, nanoclusters, quantum dots, polymer particles, hydroxyapatite (HAp) particles, fluorescent dye particles, and magnetic particles.

An example of the metal nanoparticle may include a gold nanoparticle. Examples of the fluorescent dye particle may include: cyanine dyes (e.g., Cy3, Cy5, and Cy7); xanthene dyes (e.g., fluorescein isothiocyanate (FITC), 6-carboxyfluorescein, 6-carboxy-2′,4′,7,4,7-hexachlorofluorescein (HEX), 6-carboxy-X-rhodamine (ROX or R), 5-carboxyrhodamine 6G (R6G5 or G5), 6-carboxyrhodamine-6G (R6G6 or G6), and rhodamine 110, among other fluorescein and rhodamine dyes); benzimidazole dyes (e.g., Hoechst 33258); and phenanthridine dyes (e.g., Texas Red). The diameter of the particle may range from about 5 nm to about 20 nm. If the diameter of the particle exceeds the above range, introducing the particle at the nick site may be difficult, and if the diameter is below the range, observation under SEM may be difficult.

In an embodiment, the second step may include: contacting the nicked double-stranded DNA with a DNA polymerase in the presence of at least one nucleotide conjugated to a first binding moiety to generate a modified double-stranded DNA having a first binding moiety; and contacting the modified double-stranded DNA with a detectable label conjugated to a second binding moiety to generate labeled double-stranded DNA. The second binding moiety may form a binding pair with the first binding moiety.

In an embodiment, the first binding moiety or the second binding moiety may be any one selected from biotin, streptavidin, digoxin, neutravidin, and avidin. For example, the first and second binding moieties may form a binding pair of biotin/streptavidin, biotin/neutravidin, or biotin/avidin.

A nucleotide conjugated to a first binding moiety according to an embodiment may be biotin-dUTP, and the detectable label conjugated to the second binding moiety may be a streptavidin-coated detectable label.

For example, the DNA polymerase may have 5′->3′ exonuclease activity. The DNA polymerase may remove the original nucleotide strand in the 5′->3′ direction and simultaneously replace it with labeled nucleotides via its 5′-+3′ polymerase activity. The DNA polymerase may be DNA polymerase I.

The generating of the modified double-stranded DNA may include contacting the double-stranded DNA with at least one nucleotide conjugated to a first binding moiety, at least one nucleotide (dNTPs-dTTP, dATP, dCTP, and dGTP), a DNA polymerase, or a ligase.

In an embodiment, the third step may include staining the backbone of the labeled double-stranded DNA. The third step may include electro-staining the DNA for visualization under scanning electron microscopy (SEM).

In an embodiment, the fourth step may include passing the stained double-stranded DNA through a microchannel device. Various nucleic acid stretching methods known in the art may be used to stretch the DNA into a linear form.

In an embodiment, the fifth step may further include determining the distance between each label after imaging. The fifth step may involve imaging using SEM to detect the labeling pattern on the stretched double-stranded DNA and generate positional information for optical DNA mapping.

According to another embodiment, a scanning electron microscopy (SEM)-based DNA analysis kit may include: a DNA-binding protein capable of binding to a DNA molecule through one or more of electrostatic interaction, intercalation, and groove binding; a polymer, an anhydride thereof, or a salt thereof, which is capable of binding to the DNA-binding protein via intermolecular electrostatic attraction; and a metal cation-containing component, excluding uranium, that is capable of binding to the polymer, the anhydride thereof, or the salt thereof through interaction.

Hereinbelow, examples and comparative Examples of the present disclosure are described. However, the present disclosure is not limited to the following examples.

### EXAMPLES

### Materials

Unless otherwise specified, the following materials were used.

Polyvinylpyrrolidone (PVP, 40 kDa), branched polyethyleneimine (PEI, 25 kDa), poly(allylamine hydrochloride) (PAH, 50 kDa), and polyaniline (PANI, 15 kDa) were purchased from Merck Millipore (Burlington, MA). La(OAc)₃ and LaCl₃ were purchased from Sigma-Aldrich. Qdot 585 streptavidin conjugate was purchased from Thermo Fisher Scientific (Waltham, MA). SU-8 photoresists (2005, 2015) and SU-8 developer were purchased from Kayaku Advanced Materials (Westborough, MA). PDMS and its curing agent were purchased from K1 Solution (Gwangmyeong, Korea). Ethanol (99.9%), methanol, sulfuric acid, and hydrogen peroxide were purchased from Jin Chemical (Siheung, Korea). Glacial acetic acid was purchased from Duksan Reagents (Ansan, Korea). N-trimethoxysilylpropyl-N,N,N-trimethylammonium chloride (Q-siloxane) in a 50% methanol solution was purchased from Gelest (Morrisville, PA).

### Silicon Substrate Surface-Modified to Have Positive Charge

A silicon wafer with a 30 nm-thick silicon oxide layer was purchased from Wafer Market (Yongin, Korea). The silicon wafer or glass coverslip was placed on a Teflon rack and immersed in a piranha etching solution (30:70 v/v H₂O₂/H₂SO₄) for 3 hours. Afterward, the wafer and coverslip were thoroughly rinsed with deionized water, and pH 7 test paper was used to confirm that the surface was neutral (pH 7). Next, the wafer and coverslip were sonicated in deionized water for 30 minutes, then rinsed again to expose the piranha-treated surface. To prepare a 1.1 mM solution, 150 μL of Q-siloxane (N-trimethoxysilylpropyl-N,N,N-trimethylammonium chloride) was added to 250 mL of 50% methanol in deionized water. The wafer and coverslip were incubated in this solution at 65° C. and 100 rpm for 16 hours. Finally, the substrates were rinsed three times with 99.9% ethanol and stored in 99.9% ethanol, thereby producing a silicon substrate surface-modified to have positive charge.

### PDMS Microchannel Device

The PDMS microchannel device was fabricated as described in “Counting DNA molecules on a microchannel surface for quantitative analysis” Talanta 2023, 252:123826.

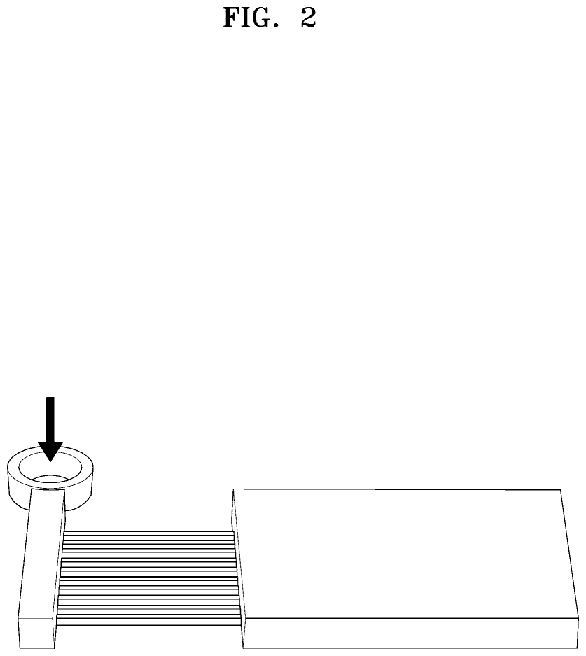

The PDMS device was manufactured using standard soft lithography replica molding techniques. First, a two-layer microchannel template was formed on a silicon wafer using repeated photolithography procedures, following the protocol described in the SU-8 2000 datasheet provided by Kayaku Advanced Materials. Subsequently, SU-8 2015 photoresist was spin-coated onto the wafer at a thickness of 20 μm using a spin coater (Midas System SPIN-1200D, Daejeon, Korea). The coated wafer was exposed to 350 nm radiation through a mask using an aligner (Midas System MDA-400LJ, Daejeon, Korea). Then, the wafer was baked and developed using an SU-8 developer, and the second layer was precisely patterned using a maskless lithography system (SmartPrint, SmartForce Technologies, La Tronche, France). Since SmartPrint is compatible with g-line photoresists, SU-8 TF 6002 was used. After preparing the template wafer, the wafer bearing the microchannel template was coated with a 10:1 (w/w) mixture of PDMS pre-polymer and curing agent and incubated at 65° C. for 12 hours. After curing, the PDMS was peeled off from the wafer, and channels were physically punched into the PDMS to form chambers. Next, the PDMS microchannel was oxidized at 100 W for 30 seconds using an air plasma generator (Femto Science Cute Basic, Korea). Finally, the PDMS device was rinsed and stored in deionized water, completing the fabrication of the PDMS microchannel device (100 μm×2.4 μm) as shown in FIG. 2.

### DNA Samples

λ phage DNA (48.5 kb), Nb.BssSI, circular double-stranded M13mp18 DNA (7.2 kb), and DNA polymerase I were purchased from New England Biolabs (Ipswich, MA).

### FE-SEM Instrument

A field-emission scanning electron microscope (FE-SEM, model JSM-7100F, JEOL) was used to observe DNA images at various magnifications under an accelerating voltage of 15 kV.

### [Metal Cation Composition]

### Comparative Example 1: La³⁺ Complex Composition @ pH 6 (La(OAc)₃ Precursor)

A solution containing 200 μM La(OAc)₃ at pH 6 was prepared by dissolving 99.9% powdered La(OAc)₃ in deionized water as a metal cation precursor.

### Comparative Example 2: La³⁺ Complex Composition @ pH 8 (La(OAc)₃ Precursor)

A solution containing 200 μM La(OAc)₃ at pH 8 was prepared by dissolving 99.9% powdered La(OAc)₃ in deionized water as a metal cation precursor.

### Comparative Example 3: La³⁺ Complex Composition @ pH 6 (LaCl₃ Precursor)

A solution containing 200 μM LaCl₃ at pH 6 was prepared by dissolving 99.9% powdered LaCl₃ in deionized water as a metal cation precursor.

### Comparative Example 4: DNA-La³⁺ Complex Composition @ pH 8 (LaCl₃ Precursor)

A solution containing 200 μM LaCl₃ at pH 8 was prepared by dissolving 99.9% powdered LaCl₃ in deionized water as a metal cation precursor.

### DNA-Polymer (or Salt) Complex Composition

### Comparative Example 5: DNA-0.5 wt % PEI Complex Composition

25 μg of λ phage DNA (48.5 kb) was diluted in 1×TE (Tris-EDTA) buffer solution (Tris 10 mM, EDTA 1 mM, pH 8.0). Subsequently, a 0.5 wt % aqueous solution of branched polyethyleneimine (PEI, 25 kDa) was added in an equal volume to the diluted buffer solution and reacted for approximately 5 minutes to prepare the DNA-PEI complex composition.

### Comparative Example 6: DNA-5 wt % PEI Complex Composition

Except that a 5 wt % aqueous solution of branched polyethyleneimine (PEI, 25 kDa) was added in an equal volume to the diluted buffer solution, the DNA-PEI complex composition was prepared in the same manner as in Comparative Example 5.

### Comparative Example 7: DNA-0.5 wt % PAH Complex Composition

Except that a 0.5 wt % aqueous solution of poly(allylamine hydrochloride) (PAH, 50 kDa) was added in an equal volume to the diluted buffer solution, the DNA-PAH complex composition was prepared in the same manner as in Comparative Example 5.

### Comparative Example 8: DNA-5 wt % PAH Complex Composition

Except that a 5 wt % aqueous solution of poly(allylamine hydrochloride) (PAH, 50 kDa) was added in an equal volume to the diluted buffer solution, the DNA-PAH complex composition was prepared in the same manner as in Comparative Example 5.

### Comparative Example 9: DNA-0.05 wt % PANI Complex Composition

Except that a 0.05 wt % aqueous solution of polyaniline (PANI, 15 kDa) was added in an equal volume to the diluted buffer solution, the DNA-PANI complex composition was prepared in the same manner as in Comparative Example 5.

### Comparative Example 10: DNA-0.5 wt % PANI Complex Composition

Except that a 0.5 wt % aqueous solution of polyaniline (PANI, 15 kDa) was added in an equal volume to the diluted buffer solution, the DNA-PANI complex composition was prepared in the same manner as in Comparative Example 5.

Preparation of DNA-Binding Protein-Truncated TALE (tTALE)—mNeongreen

The tTALE-mNeongreen plasmid was prepared according to the method described in “21 Fluorescent Protein-Based DNA Staining Dyes”, Molecules 2022, 27 (16), 5248.

First, the tTALE gene was inserted into the pET-15b vector containing two restriction sites, NdeI and XmaI. To introduce restriction sites into the tTALE sequence, forward primer (5′-ATG TTG CAT ATG GAT CTA CGC ACG CTC GGC TAC-3′) (SEQ ID NO: 2) and reverse primer (5′-ATG TTG GGA TCC ATG TTG CCC GGG GCC GCC AGA GCC GCC CCC ATG ATC CTG ACA CAA AAC AGG CAAC-3′) (SEQ ID NO: 3) were used in the PCR process. Using fluorescent protein mNeongreen as the template, a PCR product fused with the GGSGG (SEQ ID NO: 4) linker and the XmaI restriction site was generated. The bridging amino acid sequence was GGSGG (SEQ ID NO: 4). The constructed tTALE-mNeongreen plasmid was transformed into *E. coli*BL21 (DE3) strain using standard cloning procedures. The amino acid sequence of tTALE-mNeongreen is provided in Table 1 below.

TABLE 1

| Name | Amino acid sequence |
|---|---|
| Truncated | MGSSHHHHHHSSGLVPRGSHMDLRTLGYSQQQQEKIKPK |
| TALE | VRSTVAQHHEALVGHGFTHAHIVALSQHPAALGTVAVKY |
| (tTALE)- | QDMIAALPEATHEAIVGVGKQWSGARALEALLTVAGELR |
| emGFP | GPPLQLDTGQLLKIAKRGGVTAVEAVHAWRNALTGAPLN |
| | LTPAQVVAIASNNGGKQALETVQRLLPVLCQDHGLTPAQ |
| | VVAIASNGGGKQALETVQRLLPVLCQAHGLTPDQVVAIA |
| | SHDGGKQALETVQRLLPVLCQDHGLTPAQVVAIASNGGG |
| | KQALETVQRLLPVLCQAHGLTPDQVVAIASNNGGNEQAL |
| | ETVQRLLPVLCQAHGLTPAQVVAIASNGGGKQALETVQR |
| | LLPVLCQDHGGGSGGPGMVSKGEELFTGVVPILVELDGD |
| | VNGHKFSVSGEGEGDATYGKLTLKFICTTGKLPVPWPTL |
| | VTTLSWGVQCFARYPDHMKQHDFFKSAMPEGYVQERTIF |
| | FKDDGNYKTRAEVKFEGDTLVNRIELKGIDFKEDGNILG |
| | HKLEYNYFSDNVYITADKQKNGIKANFKIRHNIEDGGVQ |
| | LADHYQQNTPIGDGPVLLPDNHYLSTQSKLSKDPNEKRD |
| | HMVLLEFVTAAGITLGMDELYK (64.8 kDa) |
| | (SEQ ID NO: 1) |

### DNA-Protein-Polymer (or Salt)-Metal Cation Complex Composition

### Example 1: DNA-tTALE-mNeongreen-0.5 wt % PVP-La³⁺ Complex Composition

A mixture was prepared by combining 25 μg of λ phage DNA (48.5 kb) with 300 fmole of tTALE-mNeongreen at room temperature (25° C.) for 10 minutes. The mixture was diluted with 1×TE (Tris-EDTA) buffer solution (Tris 10 mM, EDTA 1 mM, pH 8.0), and a 0.5 wt % aqueous solution of polyvinylpyrrolidone (PVP, 40 kDa) was added in an equal volume to the diluted buffer solution and reacted for 5 minutes to obtain the DNA-tTALE-mNeongreen-PVP complex composition. Separately, a solution containing La(OAc)₃ at pH 6 was prepared by dissolving 99.9% powdered La(OAc)₃ in deionized water as a metal cation precursor. The DNA-tTALE-mNeongreen-PVP complex was attached to the substrate using a microfluidic device. After 5 minutes, the La(OAc)₃-containing solution was flowed through the same microfluidic device to prepare the DNA-tTALE-mNeongreen-PVP-La³⁺ complex composition.

### Example 2: DNA-tTALE-mNeongreen-5 wt % PVP-La³⁺ Complex Composition

Except that a 5 wt % aqueous solution of polyvinylpyrrolidone (PVP, 40 kDa) was used in an equal volume instead of the 0.5 wt % aqueous solution diluted in buffer, the DNA-tTALE-mNeongreen-PVP-La³⁺ complex composition was prepared in the same manner as in Example 1.

### Experimental Example 1: SEM-Based DNA Imaging

DNA imaging experiments using FE-SEM were conducted on the metal cation compositions from Comparative Examples 1-4, the DNA-polymer (or salt) complex compositions from Comparative Examples 5-10, and the DNA-protein-polymer (or salt)-metal cation complex composition from Example 1.

The DNA imaging experiment was performed using FE-SEM according to the following procedure.

In DNA imaging using the metal cation compositions from Comparative Examples 1-4, DNA was immobilized and stretched on a positively surface-modified silicon wafer, and after 5 minutes, the metal cation composition was injected into the arrowed region of the PDMS microchannel device (FIG. 2) to form a DNA-metal cation complex composition. The resulting sample was dried for 50 minutes and imaged using FE-SEM at a magnification of 5,000×.

DNA imaging using the DNA-polymer (or salt) complex compositions from Comparative Examples 5-10 and the DNA-protein-polymer (or salt)-metal cation complex compositions from Examples 1-2 was performed by injecting each complex into the arrowed region of the PDMS microchannel device (FIG. 2), immobilizing and stretching the DNA on a positively surface-modified silicon wafer, and drying the sample for 50 minutes. The DNA-polymer (or salt) complexes were imaged using FE-SEM at magnifications of 50,000× or 5,000×, while the DNA-protein-polymer (or salt)-metal cation complexes were imaged at 5,000×, 50,000×, or 110,000× to observe the stained DNA.

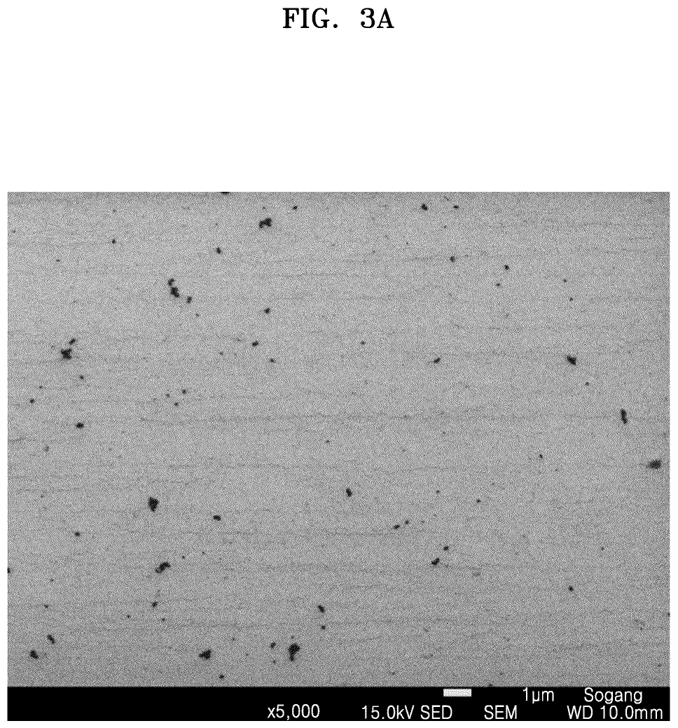

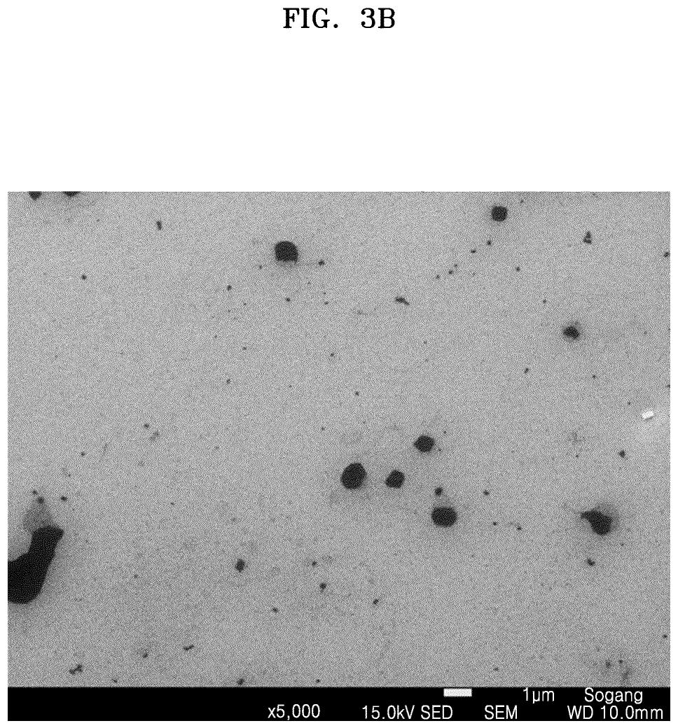

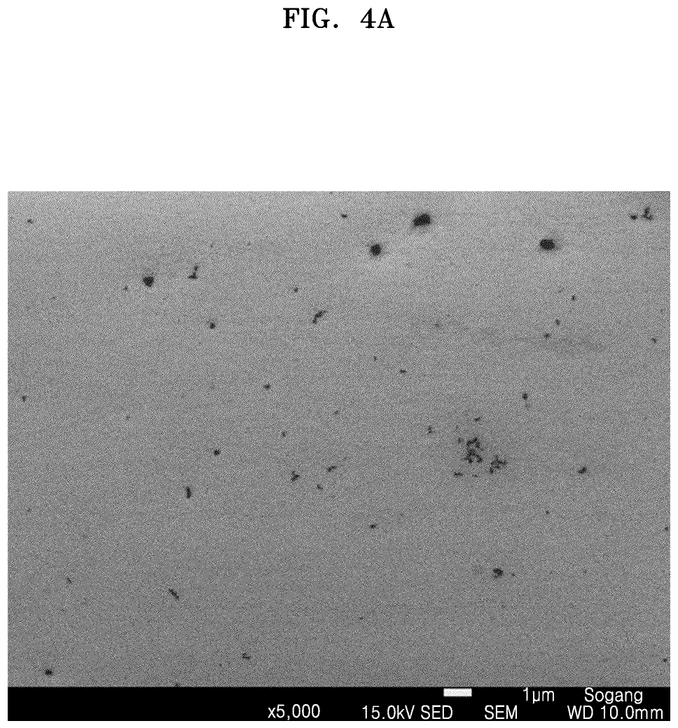

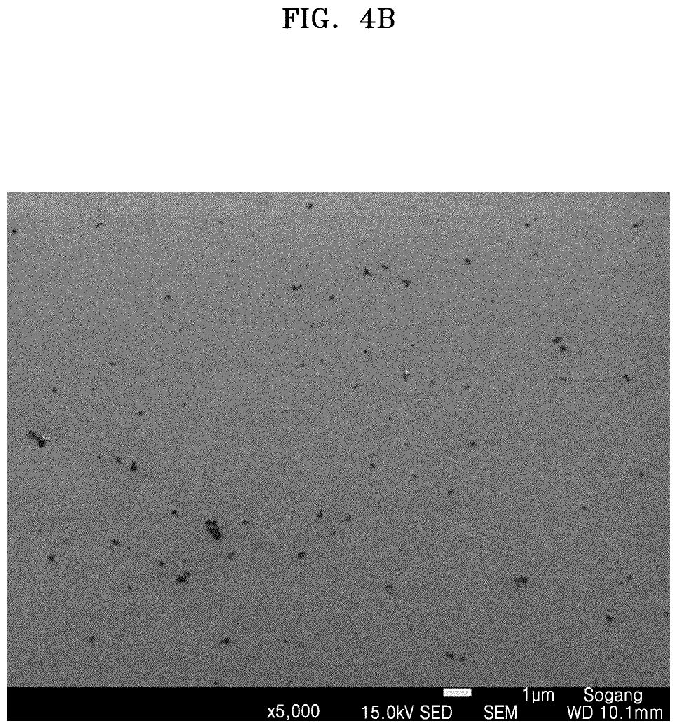

The DNA imaging results using the metal cation compositions from Comparative Examples 1-4 are shown in FIGS. 3A, 3B, 4A, and 4B, respectively.

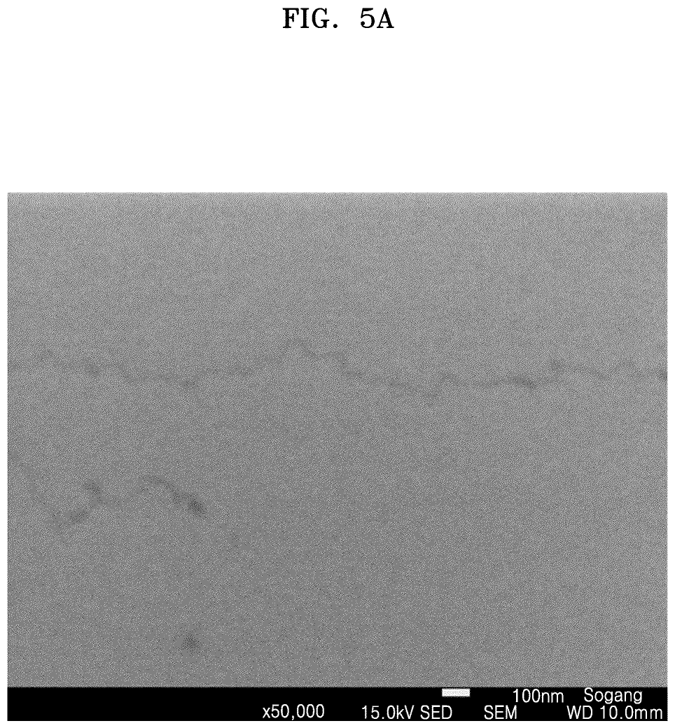

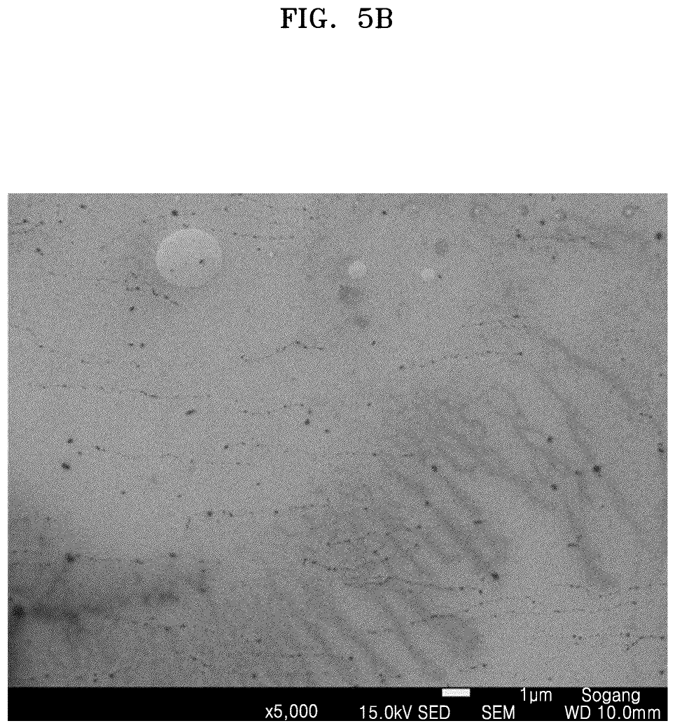

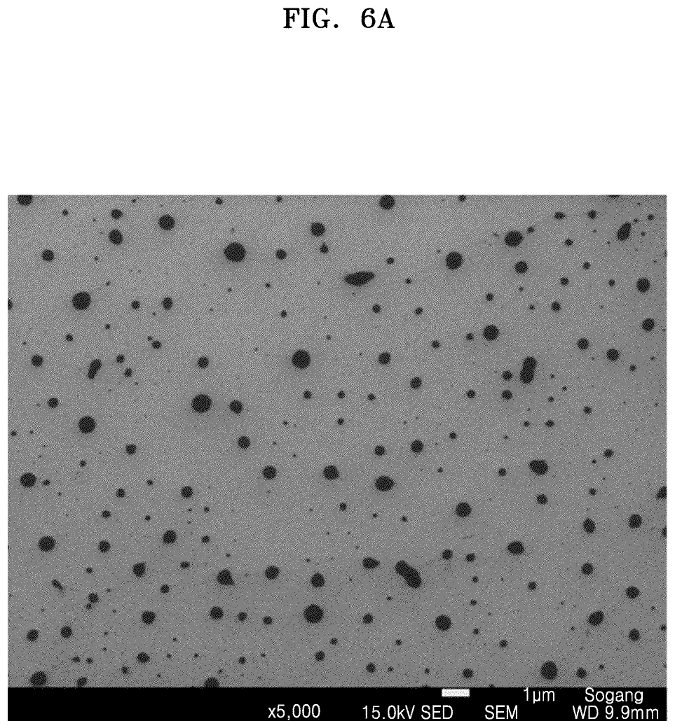

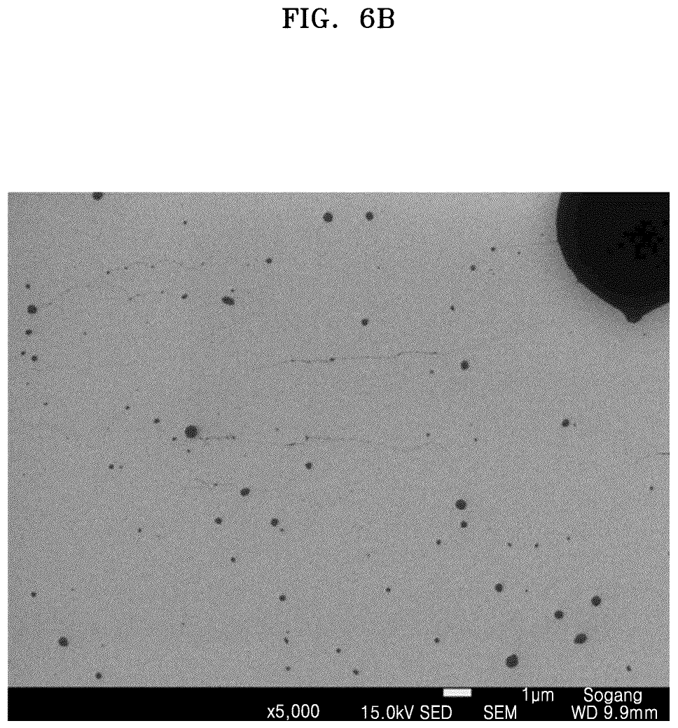

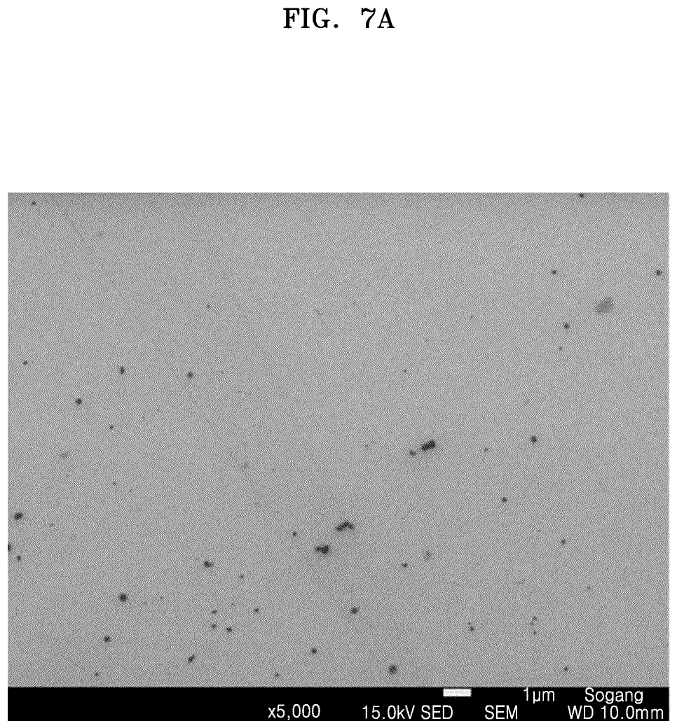

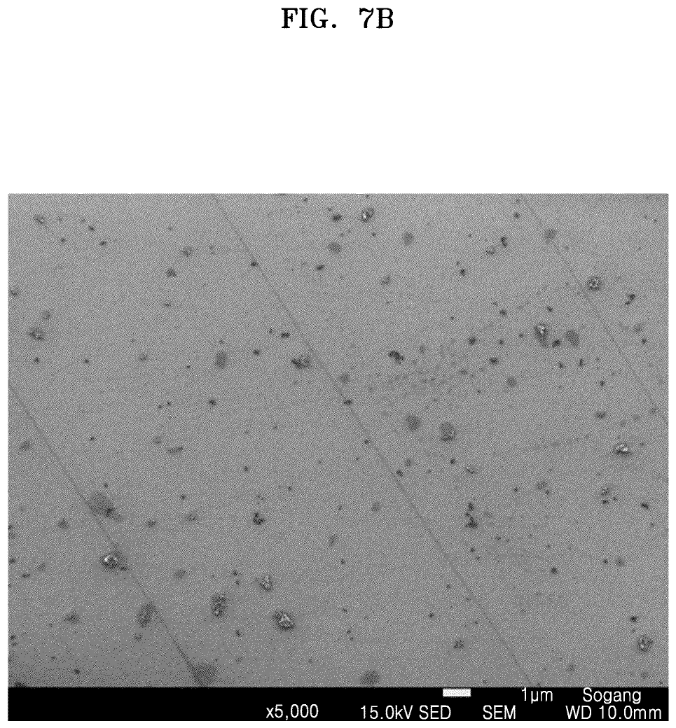

The DNA imaging of the DNA-protein-polymer (or salt)-metal cation complex compositions from Comparative Examples 5 was performed at a magnification of 50,000×, and additionally, the composition from Comparative Examples 6 to 10 were imaged at magnifications of 5,000×. The results are shown in FIGS. 5A, 5B, 6A, 6B, 7A, and 7B, respectively.

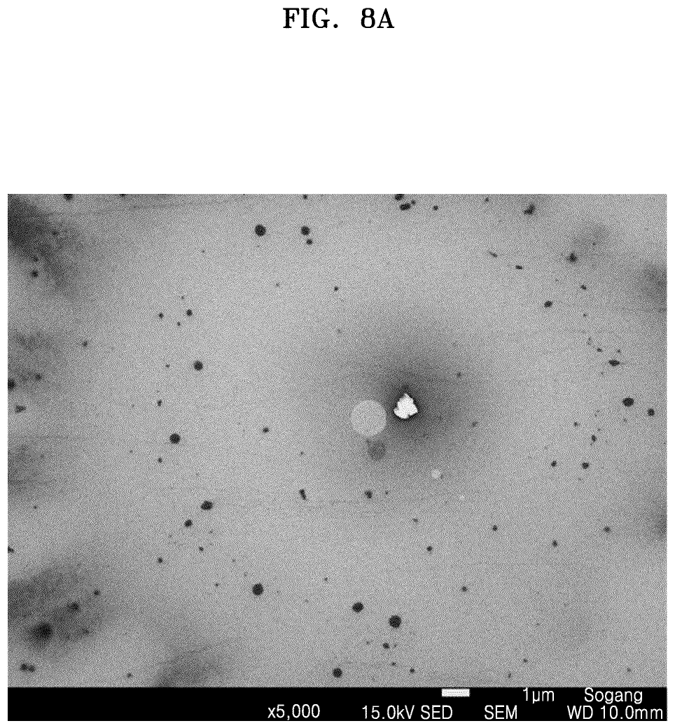

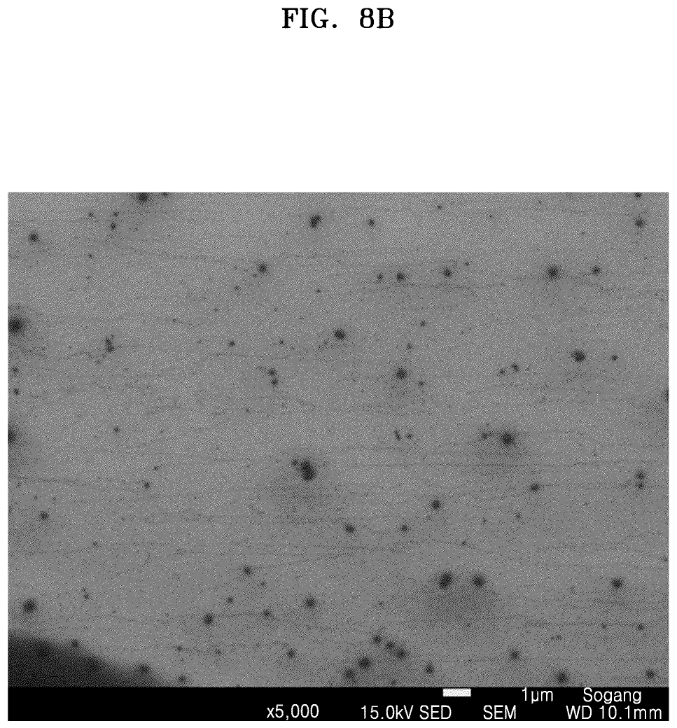

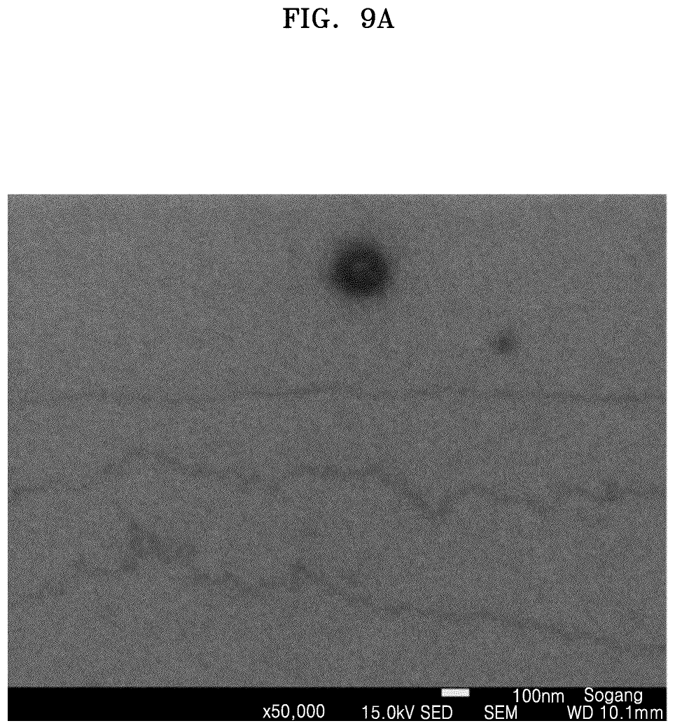

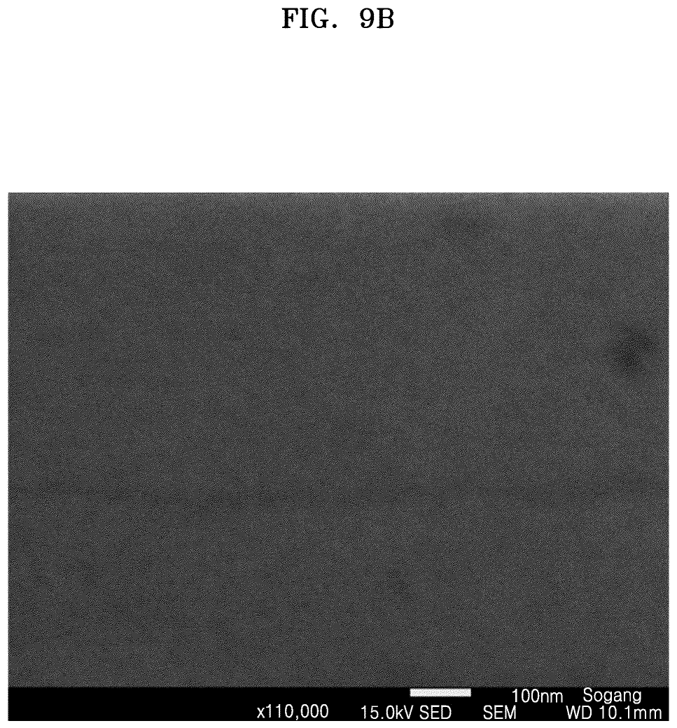

The DNA imaging of the DNA-protein-polymer (or salt)-metal cation complex compositions from Examples 1 and 2 was performed at a magnification of 5,000×. Additionally, the composition from Example 2 was imaged at magnifications of 50,000× and 110,000×. The results are shown in FIGS. 8A, 8B, 9A, and 9B, respectively.

Referring to FIGS. 3A, 3B, 4A, and 4B, the DNA-metal cation complexes prepared using the metal cation compositions from Comparative Examples 1 and 3 (@ pH 6) in FIGS. 3A and 4A showed more pronounced DNA imaging results compared to those prepared using the compositions from Comparative Examples 2 and 4 (@ pH 8) in FIGS. 3B and 4B, but they failed to produce continuous structural imaging of DNA molecules. In other words, although components mediated by the metal cations were bound to the DNA molecules, effective DNA imaging was not achieved.

Referring to FIGS. 5A, 5B, 6A, 6B, 7A, and 7B, the DNA-polymer (or salt) complex compositions (using PEI polymer) from Comparative Examples 5 and 6, shown in FIGS. 5A and 5B, exhibited relatively weak DNA imaging due to partial binding to the DNA molecules when compared with other polymers used in the other comparative examples. However, in most DNA-polymer (or salt) complex compositions, even though the DNA was pre-immobilized to the substrate, condensation of the DNA occurred due to charge interactions, making DNA imaging infeasible.

Referring to FIGS. 8A, 8B, 9A, and 9B, the DNA-protein-polymer (or salt)-metal cation complex compositions prepared in Examples 1 and 2 successfully achieved clear and continuous imaging of DNA molecular structures across a wide magnification range from 5,000× to 110,000×. Among these compositions, the DNA-protein-polymer (or salt)-metal cation complex composition prepared in Example 2 (@ pH 6, 5 wt % PVP) provided clearer and more continuous imaging of DNA molecular structures. These results are considered to be attributable to the polymer binding mediated by the DNA-binding protein and the effective attachment of metal cations to the DNA.

### Reference Example 1: Circular Double-Stranded M13mp18 DNA not Treated with Nickase

A total of 500 ng of circular double-stranded M13mp18 DNA (7.2 kb) was treated with 10 units of DNA polymerase I (New England Biolabs) to label the existing single-strand break (SSB) sites with biotin. The biotin-labeled circular double-stranded M13mp18 DNA (7.2 kb) containing SSBs was diluted in 1×TE solution. Subsequently, 30 nM streptavidin-conjugated quantum dots (Qdot 585, Thermo Fisher Scientific) were attached to the biotin-labeled SSB sites via biotin-streptavidin interaction to prepare the DNA solution.

### Example 3: Circular Double-Stranded M13mp18 DNA Treated with Nickase

A total of 500 ng of M13mp18 double-stranded DNA was incubated in NEB buffer at 16° C. for 2 hours with 20 units of Nb.BbvCI (New England Biolabs), 10 units of DNA polymerase I (New England Biolabs), and a nucleotide mixture containing biotin-labeled dUTP (Biotin-16-dUTP, Jena Bioscience), dTTP, dATP, dCTP, and dGTP. After nick translation, the DNA solution was diluted to a final concentration of 10 ng/μL. Next, the diluted DNA solution was mixed with 30 nM streptavidin-conjugated quantum dots (Qdot 585, Thermo Fisher Scientific) and incubated at room temperature for 10 minutes. To separate the DNA from unbound Qdot 585, a mixed cellulose ester membrane with a diameter of 25 mm and a pore size of 25 nm was placed over 100 μL. of 1×TE buffer, and the DNA solution was added onto the membrane and incubated for 30 minutes. The DNA solution remaining on the membrane was then carefully recovered.

### Experimental Example 2: SEM-Based DNA Detection Using QD Labeling

QD (quantum dot)-labeled circular M13mp18 DNA solutions from Reference Example 1 and Example 3 were injected into the arrowed region of the PDMS microchannel device (FIG. 2) along with the DNA-tTALE-mNeongreen-0.5 wt % PVP-La³⁺ complex composition prepared in Example 1, and the circular M13mp18 DNA was immobilized and stretched on a positively surface-modified silicon wafer. Before removing the PDMS microchannel device from the silicon wafer, the QD-labeled circular M13mp18 DNA from Reference Example 1 and Example 3 was dried for 50 minutes, and the resulting sample was examined using FE-SEM at a magnification of 50,000× to detect DNA.

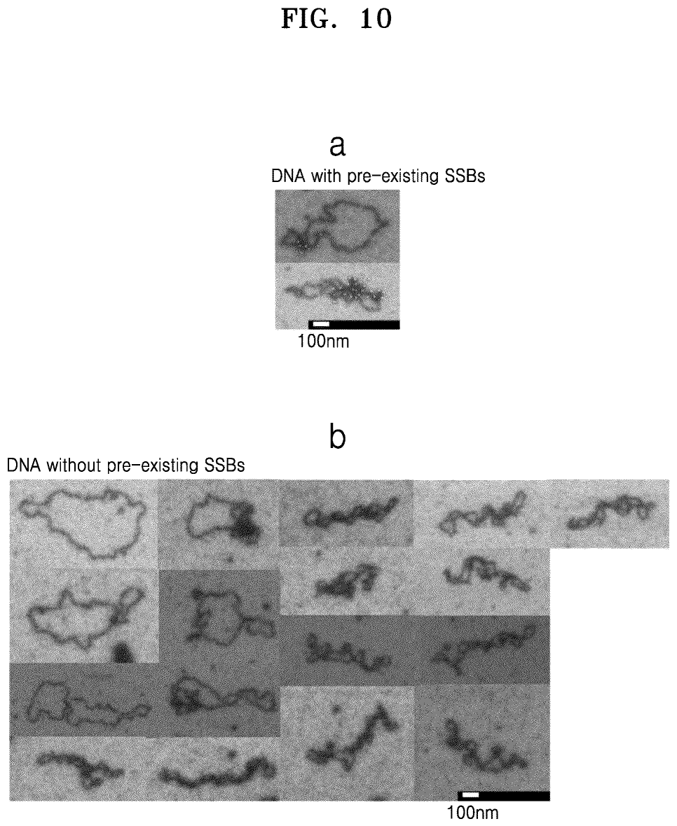

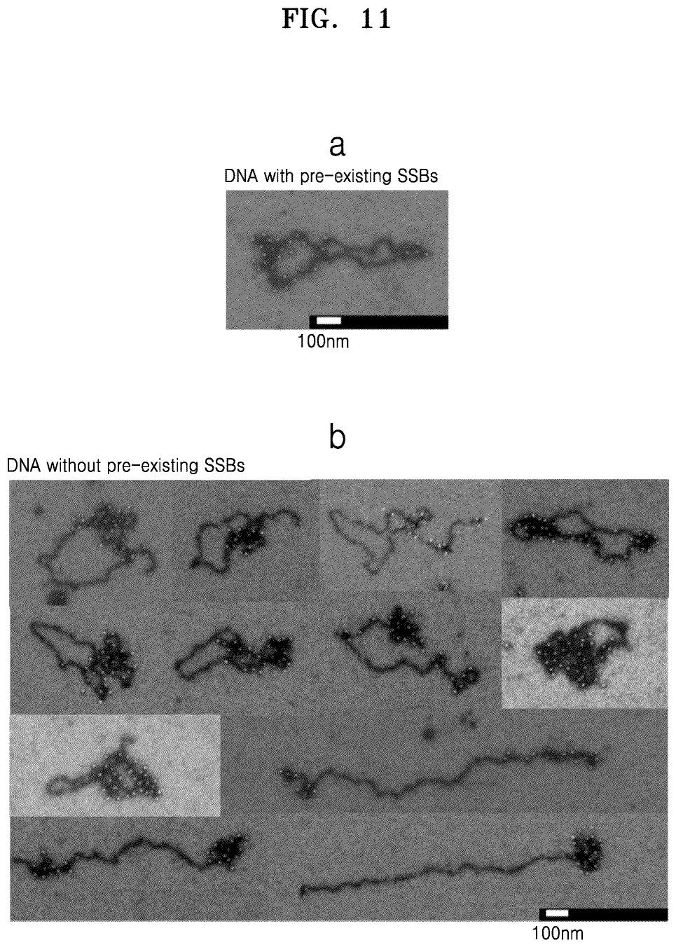

The results of Reference Example 1 are shown in FIG. 10 (A: DNA with pre-existing SSBs, B: DNA without pre-existing SSBs). The results of Example 3 are shown in FIG. 11 (A: DNA with pre-existing SSBs, B: DNA without pre-existing SSBs).

Referring to FIG. 10, among 19 circular M13mp18 DNA molecules, 17 circular M13mp18 DNA molecules were not QD-labeled and 2 circular M13mp18 DNA molecules were QD-labeled. This result indicates that only a subset of the circular M13mp18 DNA molecules contained pre-existing SSBs.

Referring to FIG. 11, 12 out of 13 circular M13mp18 DNA molecules exhibited QD labeling confined to a single broad region due to the proximity of two nick sites on the opposite strand, indicating the absence of pre-existing SSBs. Among these DNA molecules, three linearized M13mp18 DNA molecules with QDs attached at both ends were observed. This is presumed to result from the cleavage of M13mp18 DNA due to the close spacing of the nick sites. One circular M13mp18 DNA molecule, similar to the DNA in Reference Example 1 that was not treated with nickase, showed QD labeling in two separate regions, allowing detection of DNA containing pre-existing SSBs.

According to an embodiment of the present disclosure, the DNA-protein-polymer-metal cation complex enables high-resolution, real-time imaging of DNA molecules with diverse shapes and lengths at high magnification using SEM, thereby improving DNA detection and enhancing the precision of DNA optical mapping.

Thus far, exemplary embodiments have been described and illustrated in the accompanying drawings to aid in the understanding of the present disclosure. However, it should be understood that these embodiments are provided for illustrative purposes only and do not limit the disclosure. It should also be understood that the disclosure is not limited to the descriptions and drawings presented herein, as various other modifications may occur to those of ordinary skill in the art.

According to an aspect of the present disclosure, the DNA-protein-polymer-metal cation complex of the present disclosure includes, in addition to the DNA-binding protein, a polymer, its anhydride or salt, and a metal cation-containing component excluding uranium. When imaging DNA molecules using a scanning electron microscope (SEM), the metal cations in the DNA-protein-polymer complex block secondary electron emission, enabling high-resolution, real-time imaging of DNA molecules of various shapes and lengths even at high magnifications. Furthermore, the DNA-protein-polymer-metal cation complex enhances DNA detection, thereby improving the accuracy of DNA optical mapping.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A DNA-protein-polymer-metal cation complex comprising:
a DNA molecule;
a DNA-binding protein comprising a peptide, the peptide having an amino acid sequence capable of binding to the DNA molecule and having at least one functional group at at least one of the N-terminal and C-terminal of the peptide;
a polymer, an anhydride thereof, or a salt thereof, which is capable of binding to the DNA-binding protein via intermolecular electrostatic interaction; and
a metal cation-containing component, excluding uranium, that is capable of binding to the polymer, an anhydride thereof, or a salt thereof through interaction.

2. The DNA-protein-polymer-metal cation complex of claim 1,
wherein the polymer comprises at least one atom selected from oxygen and nitrogen,
wherein the at least one atom is capable of forming a hydrogen bond with a hydrogen atom of an O—H or N—H group present in the DNA-binding protein.

3. The DNA-protein-polymer-metal cation complex of claim 1,
wherein the polymer, an anhydride thereof, or a salt thereof comprises at least one structural unit represented by any one of Formulae 1 to 12, and has a weight average molecular weight of about 10 kilodaltons to about 100 kilodaltons: 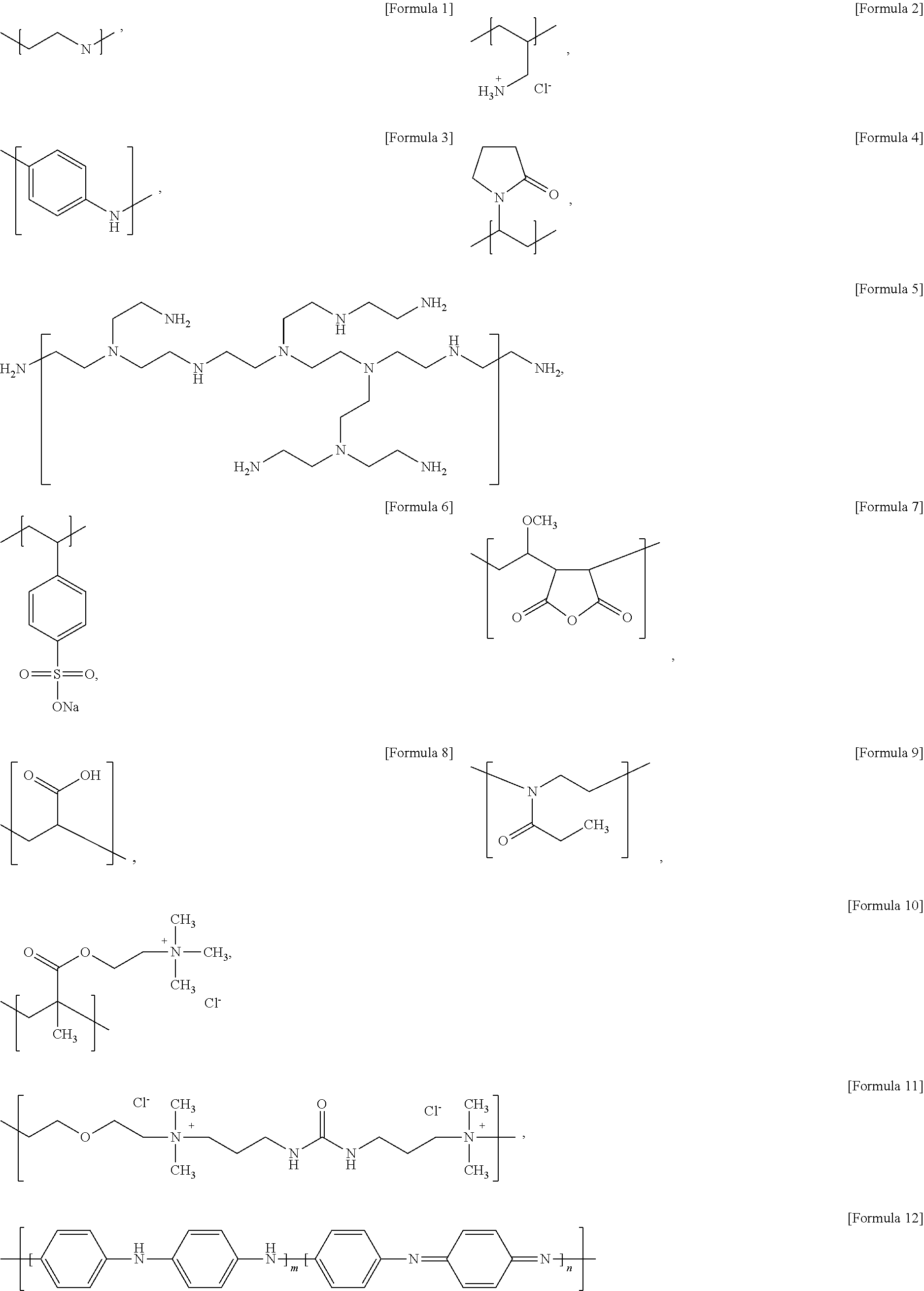
wherein in Formula 12,
m+n is 1.

4. The DNA-protein-polymer-metal cation complex of claim 1,
wherein the DNA molecule has a shape selected from linear, circular, helical, supercoiled, Christmas tree-shaped, double-stranded, or single-stranded.

5. The DNA-protein-polymer-metal cation complex of claim 1,
wherein the DNA-binding protein further comprises a fluorescent protein.

6. The DNA-protein-polymer-metal cation complex of claim 1,
wherein the DNA molecule and the DNA-binding protein are capable of binding through one or more of electrostatic interaction, intercalation, and groove binding.

7. The DNA-protein-polymer-metal cation complex of claim 1,
wherein the metal cation has a valence of +2, +3, +4, +5, or +6, and
comprises at least one metal selected from lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), hafnium (Hf), tantalum (Ta), tungsten (W), rhenium (Re), osmium (Os), iridium (Ir), platinum (Pt), and gold (Au).

8. The DNA-protein-polymer-metal cation complex of claim 1,
wherein the metal cation comprises La³⁺, Eu²⁺, Eu³⁺, Gd³⁺, Ga⁴⁺, Dy³⁺, or a combination thereof.

9. The DNA-protein-polymer-metal cation complex of claim 1,
wherein the complex is configured to stain DNA having a two-dimensional or three-dimensional structure for visualization by scanning electron microscopy (SEM).

10. The DNA-protein-polymer-metal cation complex of claim 1,
wherein the complex is configured to enable visualization of continuous DNA morphology by scanning electron microscopy (SEM).

11. A method of DNA imaging using a DNA-protein-polymer-metal cation complex, the method comprising:
providing a silicon substrate surface-modified to have a positive charge;
immobilizing and stretching DNA on a surface of the silicon substrate using a PDMS microchannel device into which the composition including claim 1 is injected; and
removing the PDMS microchannel device from the silicon substrate and imaging the immobilized and stretched DNA using a scanning electron microscope (SEM).

12. A scanning electron microscopy (SEM)-based method for detecting DNA using a DNA-protein-polymer-metal cation complex, comprising:
a first step of forming a nick in a target sequence motif by contacting double-stranded DNA with a nickase;
a second step of generating labeled double-stranded DNA by incorporating a labeled nucleotide into the nick site of the nicked double-stranded DNA;
a third step of staining the double-stranded DNA by contacting the labeled double-stranded DNA with a DNA-binding protein and a polymer, an anhydride thereof, or a salt thereof, and a metal cation-containing component excluding uranium;
a fourth step of stretching and immobilizing the stained double-stranded DNA on a substrate having a functionalized surface; and
a fifth step of imaging the immobilized double-stranded DNA using a scanning electron microscope.

13. The DNA detection method of claim 12,
wherein the labeled nucleotide is a nucleotide directly or indirectly conjugated to a detectable label.

14. The DNA detection method of claim 12,
wherein the detectable label comprises at least one label selected from metal nanoparticles, oxide particles, sulfide particles, nanoclusters, quantum dots, polymer particles, hydroxyapatite (HAp) particles, fluorescent dye particles, and magnetic particles.

15. The DNA detection method of claim 12,
wherein the DNA-binding protein comprises a fluorescent protein.

16. The DNA detection method of claim 12,
wherein the polymer, an anhydride thereof, or a salt thereof comprises at least one structural unit represented by any one of Formulae 1 to 12, and has a weight average molecular weight ranging from 10 kilodaltons to 100 kilodaltons: 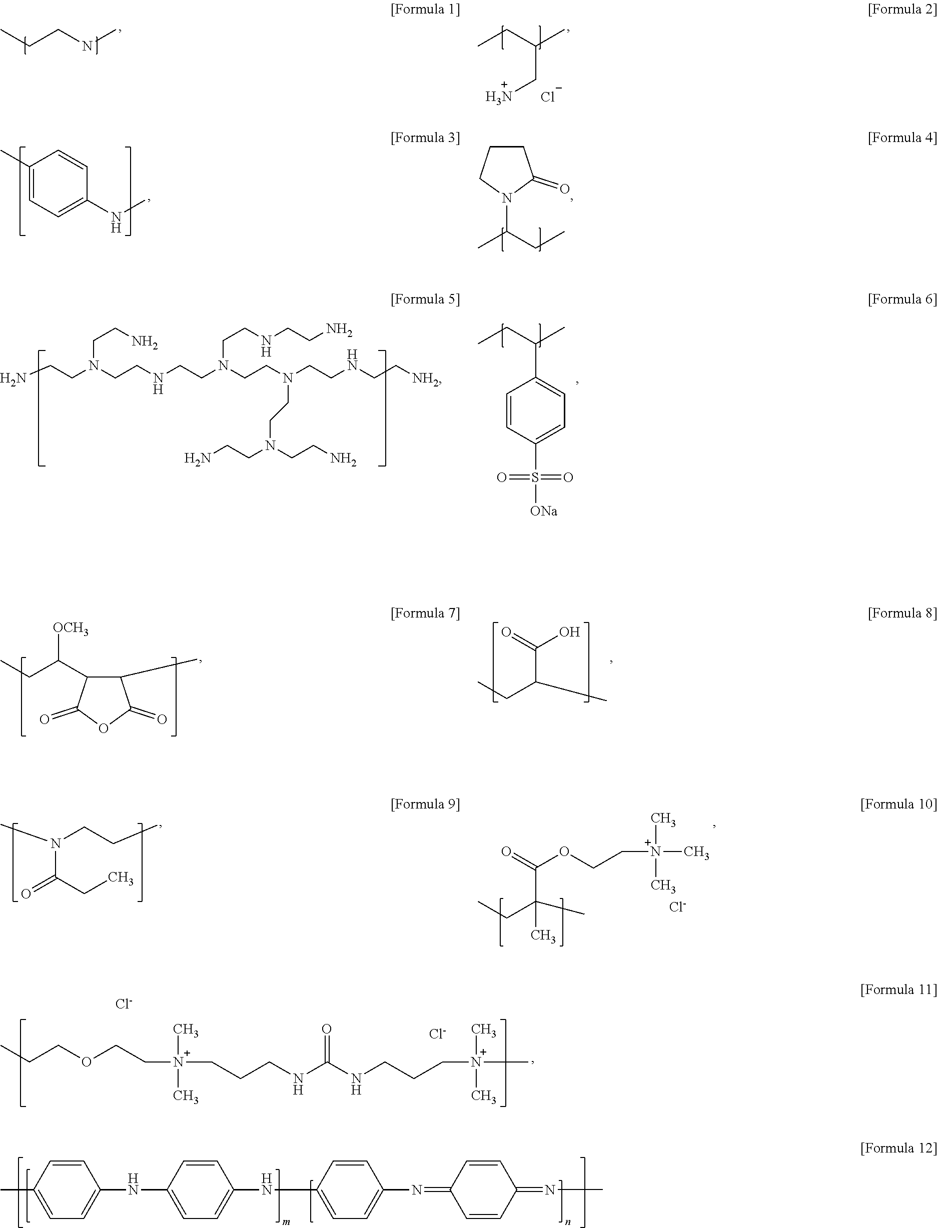
wherein in Formula 12,
m+n is 1.

17. The DNA detection method of claim 12,
wherein the metal cation comprises La³⁺, Eu²⁺, Eu³⁺, Gd³⁺, Ga⁴⁺, Dy³⁺, or a combination thereof.
